(19) United States
(12) Patent Application Publication
(10) Pub. No.: US 2022/0160365 A1
(43) Pub. Date: May 26, 2022

(54) LIGATION DEVICE WITH A PROTRUSION AND NOTCH FOR LOCKING THE LIGATION DEVICE

(71) Applicant: RECOVERY SCIENCE, LLC, Rockville, MD (US)

(72) Inventors: Shalin Nayan SHAH, Rockville, MD (US); Kusum Rasiklal SHAH, Rockville, MD (US)

(21) Appl. No.: 17/425,813

(22) PCT Filed: Jan. 28, 2020
(86) PCT No.: PCT/US2020/015454

§ 371 Date: Jul. 26, 2021

Related U.S. Application Data
(60) Provisional application No. 62/918,423, filed on Jan. 28, 2019.

(51) Int. Cl. A61B 17/12

(52) U.S. Cl. CPC A61B 17/12009; A61B 2017/00818; A61B 2017/12018

(57) ABSTRACT

A ligation device for treating hemorrhoids includes a locking mechanism. The ligation device includes an inner tubular member having a first end, a second end, and a protrusion on an outer surface. The first end includes a first opening and the second end includes a second opening. Further, the ligation device includes an outer tubular member having an inner configuration corresponding to that of the inner tubular member. The outer tubular member including a notch such that a reception of the protrusion of the inner tubular member by a first portion of the notch of the outer tubular member allows vertical movement of the outer tubular member and a reception of the protrusion of the inner tubular member by a second portion of the notch of the outer tubular member prevents vertical movement of the outer tubular member towards the first end of the inner tubular member.

### CROSS-REFERENCE TO RELATED APPLICATIONS

This application claims priority to and the full benefit of U.S. Provisional Application Ser. No. 62/918,423, filed Jan. 28, 2019, and titled “Hemorrhoid Ligator,” the entire contents of which is incorporated herein by reference.

### FIELD OF THE INVENTION

The invention relates to a ligation device including a protrusion and notch for locking the ligation device.

### BACKGROUND OF THE INVENTION

Ligation devices have been used to treat hemorrhoids. However, such ligation devices fail to prevent unintended movement of an outer tubular member towards a front end of an inner tubular member during treatment, which can result in early deployment of an elastic band, failure to ligate, or improper or inadequate ligation. Additionally, unintended movement of an outer tubular member towards a front end of an inner tubular member during treatment may also result in potential injuries to a patient's rectum receiving treatment using such a ligation device. These and other drawbacks exist.

### SUMMARY OF THE INVENTION

An aspect of the disclosure relates to a ligation device for treating hemorrhoids, the ligation device comprising: an inner tubular member having an inner surface, an outer surface, a first end, a second end, and a protrusion on the outer surface, the first end having a first opening and the second end having a second opening; a suction member partially in the inner tubular member, the suction member including a handle portion and an end portion, and the handle portion allowing the end portion of the suction member to be pulled away from the first end of the inner tubular member; a stopper member attached to the second end of the inner tubular member, the stopper member preventing the suction member from being entirely pulled out of the inner tubular member; and an outer tubular member having an inner configuration corresponding to that of the inner tubular member, the outer tubular member including a notch that is configured to receive the protrusion of the inner tubular member, wherein a reception of the protrusion of the inner tubular member by a first portion of the notch of the outer tubular member allows the movement of the outer tubular member toward the first end of the inner tubular member and a reception of the protrusion of the inner tubular member by a second portion of the notch of the outer tubular member prevents the movement of the outer tubular member toward the first end of the inner tubular member.

Another aspect of the disclosure relates to a ligation device comprising: an inner tubular member having an inner surface, an outer surface, a first end, a second end, and a protrusion on the outer surface, the first end having a first opening and the second end having a second opening; a suction member in the inner tubular member, the suction member including a handle portion, and the handle portion allowing the suction member to be pulled toward and pushed away from the second end of the inner tubular member; and an outer tubular member having an inner configuration corresponding to that of the inner tubular member, the outer tubular member including a notch that is configured to receive the protrusion of the inner tubular member, the notch including a wider portion and a narrower portion, the narrower portion of the notch preventing movement of the outer tubular member toward the first end of the inner tubular member.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. The mere fact that certain elements are recited in mutually different dependent claims does not indicate that these elements cannot be used in combination. In addition, as used in the specification and the claims, the term “or” means “and/or” unless the context clearly dictates otherwise.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1A shows a ligation device in a locked position, in accordance with one or more embodiments.

FIG. 1B a ligation device in an unlocked position, in accordance with one or more embodiments.

FIG. 2A shows a suction member, in accordance with one or more embodiments.

FIG. 2B shows an inner tubular member, in accordance with one or more embodiments.

FIG. 2C shows an outer tubular member, in accordance with one or more embodiments.

FIG. 2D shows a stopper member, in accordance with one or more embodiments.

FIG. 2E shows a loading member, in accordance with one or more embodiments.

FIG. 2F shows a different view of the configuration of the outer tubular member in FIG. 2C, in accordance with one or more embodiments;

FIG. 2G shows another configuration of the outer tubular member, in accordance with one or more embodiments;

FIG. 3A shows a configuration of an inner tubular member and an outer tubular member in a locked position, in accordance with one or more embodiments.

FIG. 3B shows a different view of the configuration of the inner tubular member and the outer tubular member in FIG. 3A, in accordance with one or more embodiments.

FIG. 3C shows another configuration of an inner tubular member and an outer tubular member in a locked position, in accordance with one or more embodiments.

FIG. 3D shows a different view of the configuration of the inner tubular member and the outer tubular member in FIG. 3C, in accordance with one or more embodiments.

FIG. 3E shows a configuration of an inner tubular member and an outer tubular member in an unlocked position, in accordance with one or more embodiments.

FIG. 3F shows a different view of the configuration of the inner tubular member and the outer tubular member in FIG. 3E, in accordance with one or more embodiments.

FIG. 4A shows a configuration of an inner tubular member and a suction member, in accordance with one or more embodiments.

FIG. 4B shows a different angle of a configuration of an inner tubular member and a suction member, in accordance with one or more embodiments.

FIGS. 5A, 5B, 5C, and 5D show different configurations of portion A shown in FIG. 4A, in accordance with one or more embodiments.

FIG. 6A shows another ligation device in a locked position, in accordance with one or more embodiments.

FIG. 6B shows another ligation device in an unlocked position, in accordance with one or more embodiments.

FIG. 7 shows another configuration of a suction member, in accordance with one or more embodiments.

### DETAILED DESCRIPTION OF THE INVENTION

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement.

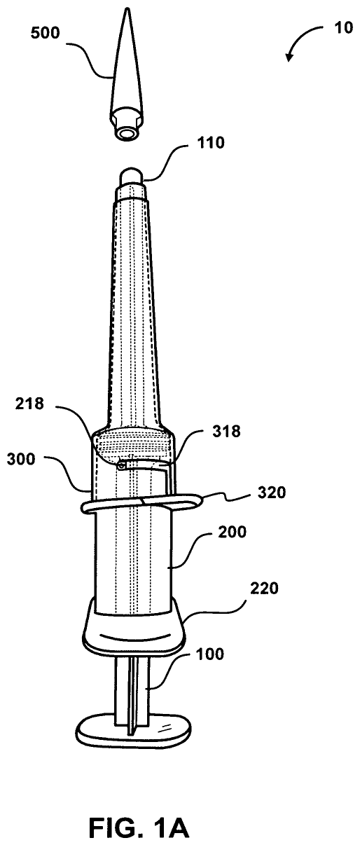

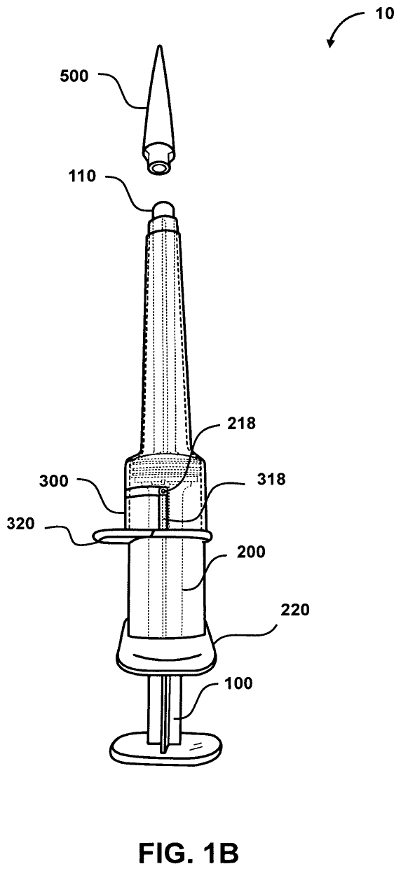

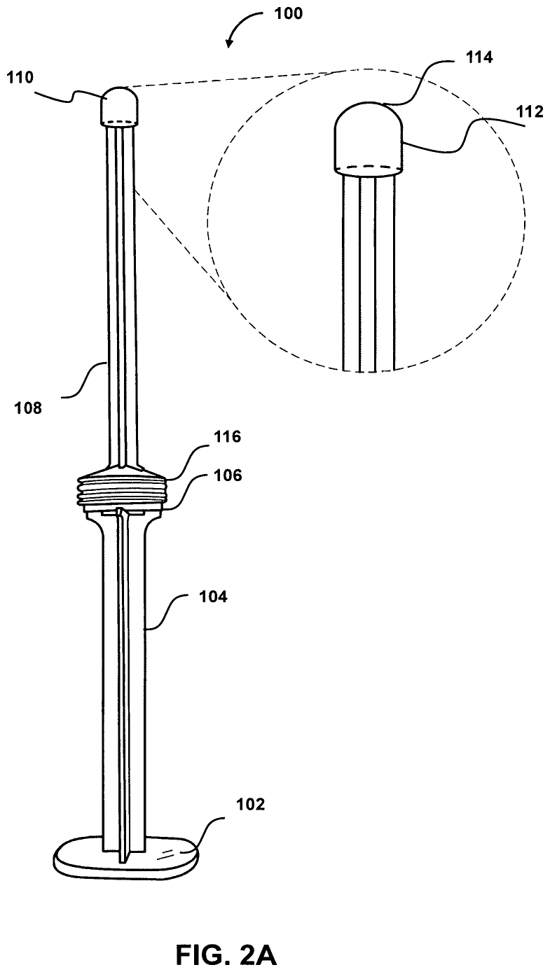

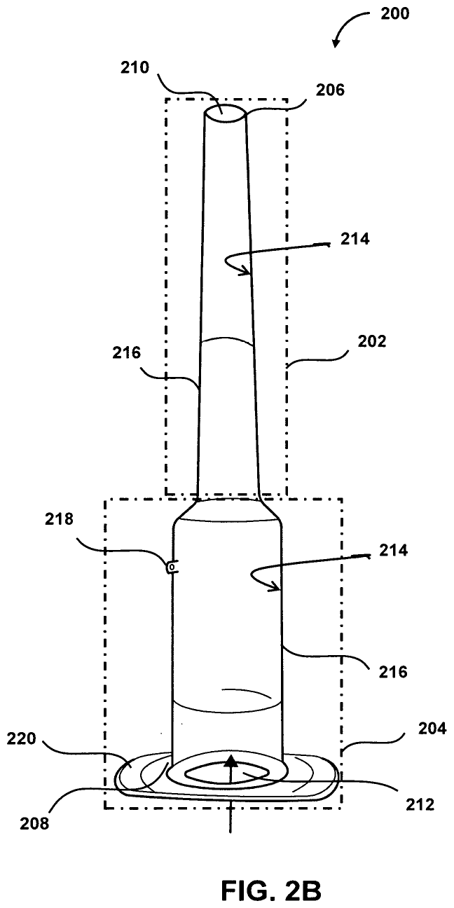

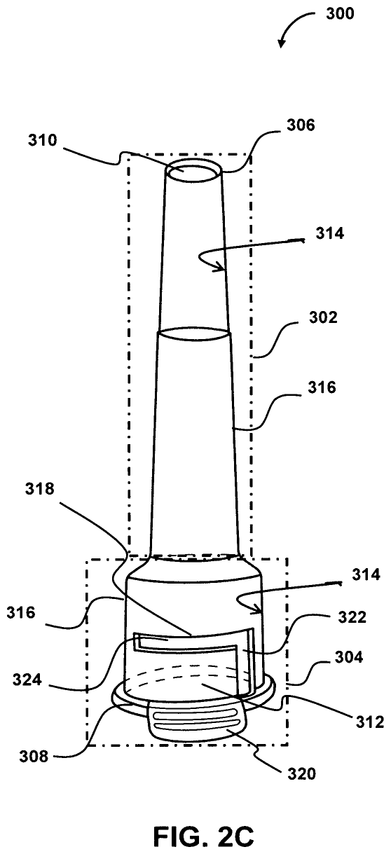

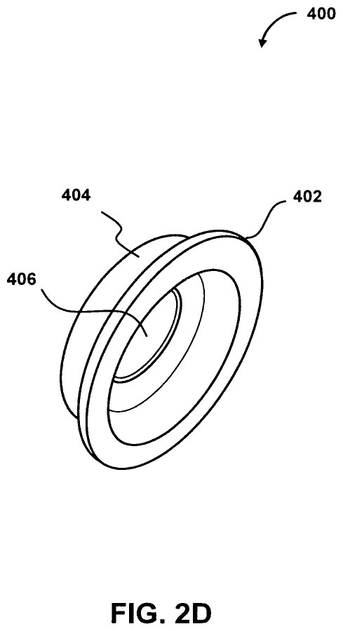

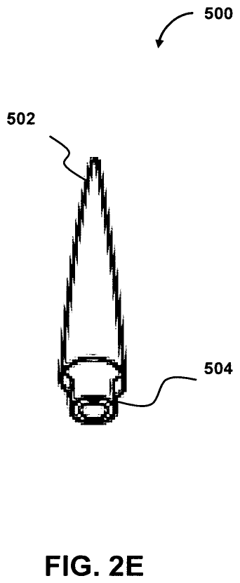

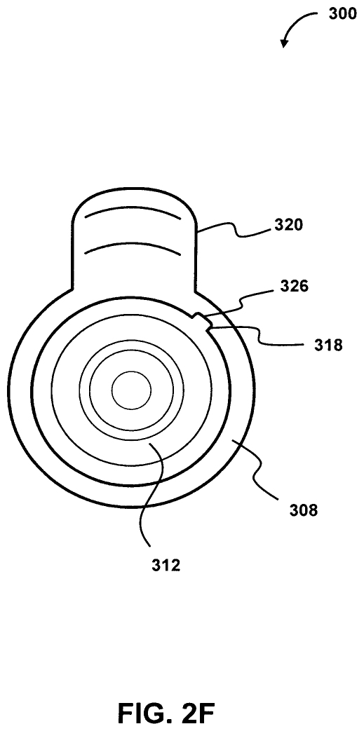

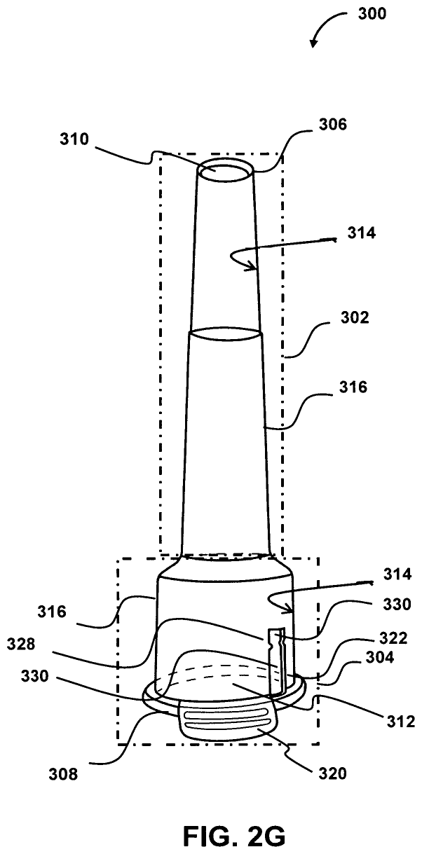

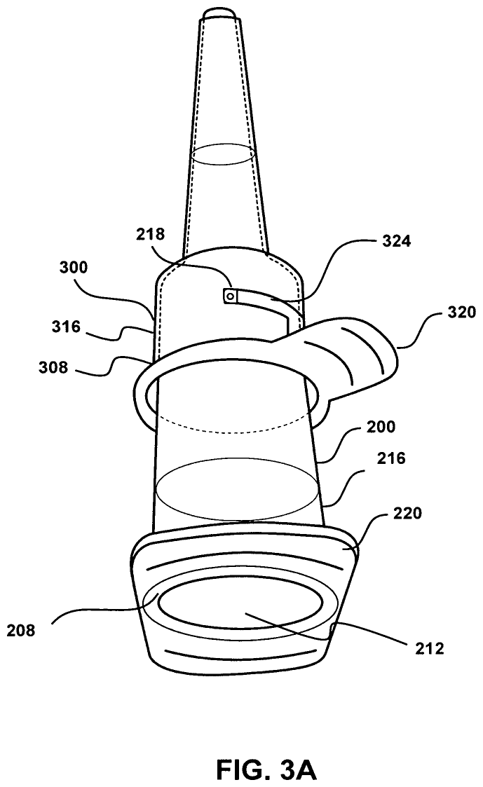

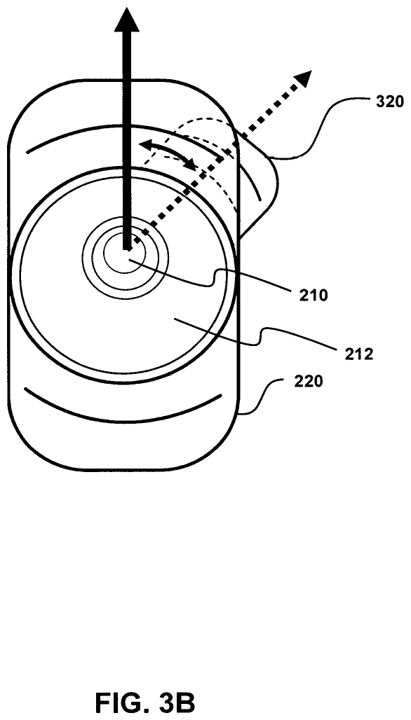

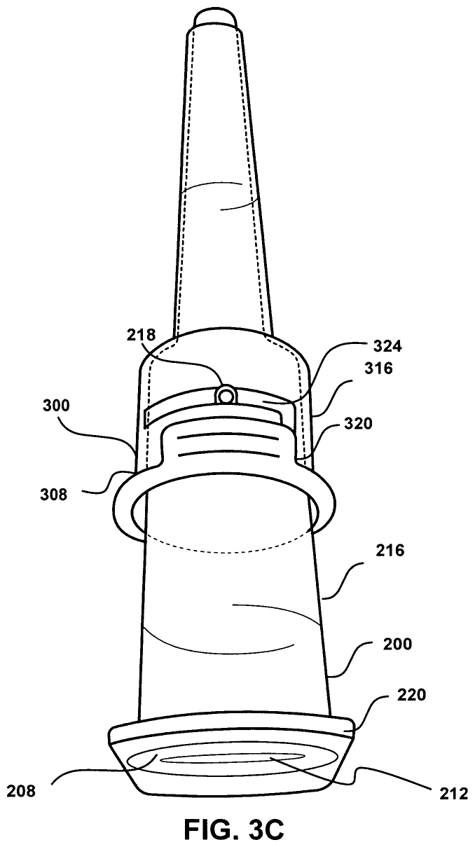

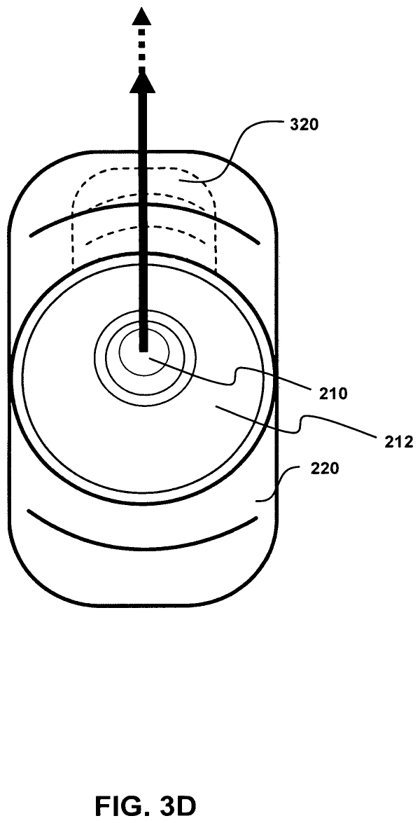

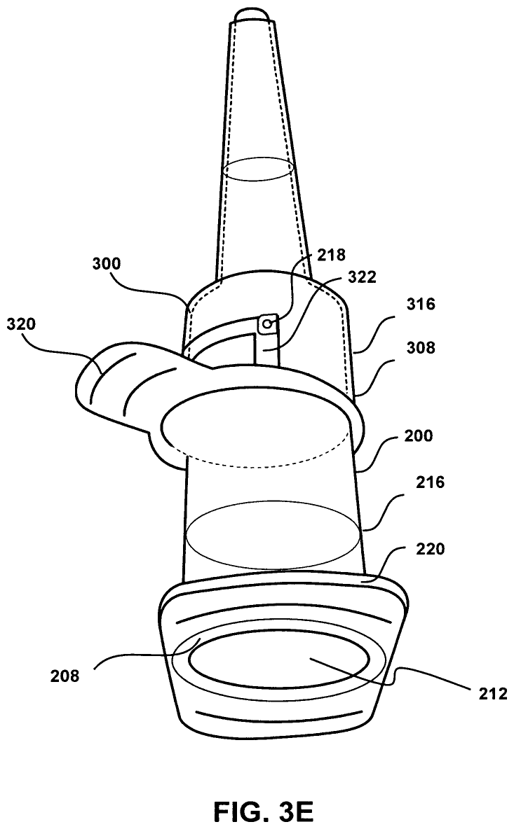

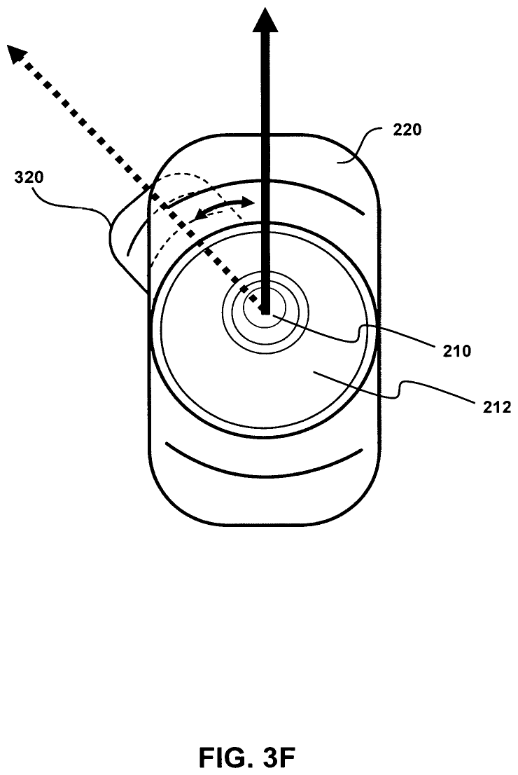

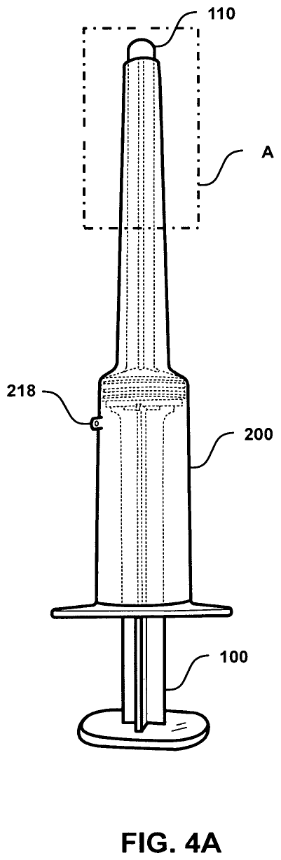

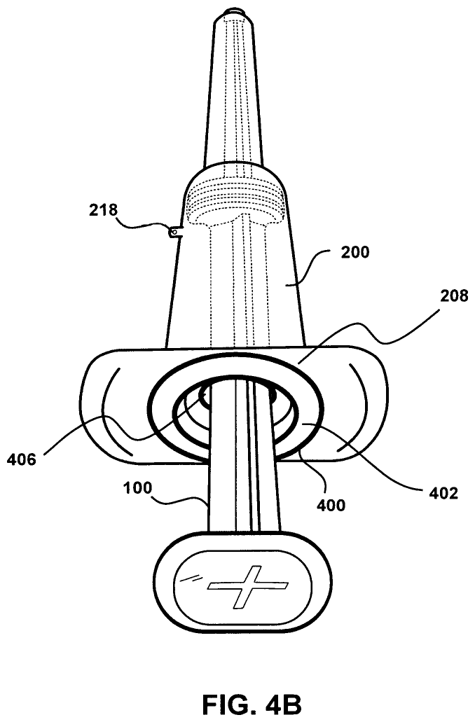

FIG. 1A illustrates a first embodiment of a ligation device 10 in a locked position and FIG. 1B illustrates the first embodiment of the ligation device 10 in an unlocked position. The ligation device 10 includes a suction member 100, an inner tubular member 200, an outer tubular member 300, a stopper member 400, and a loading member 500. FIGS. 2A, 2B, 2C, 2D, 2E, 2F, 3A, 3B, 4A, and 4B illustrate the different components of the ligation device 10. For example, FIG. 2A illustrates the suction member 100 of the ligation device 10, FIG. 2B illustrates the inner tubular member 200 of the ligation device 10, FIG. 2C illustrates the outer tubular member 300 of the ligation device 10, FIG. 2D illustrates the stopper member 400 of the ligation device 10, FIG. 2E illustrates the loading member 500 of the ligation device 10, FIG. 2F illustrates a different view of the configuration of the outer tubular member 300 in FIG. 2C, FIG. 2G shows another configuration of the outer tubular member 300, FIGS. 3A and 3B show a configuration of an inner tubular member and an outer tubular member in a locked position, FIGS. 3C and 3D show another configuration of an inner tubular member and an outer tubular member in a locked position, FIGS. 3E and 3F show a configuration of an inner tubular member and an outer tubular member in an unlocked position, FIG. 4A illustrates the suction member 100 and the inner tubular member 200 of the ligation device 10, and FIG. 4B illustrates the suction member 100, inner tubular member 200, and the stopper member 400 of the ligation device 10. In FIGS. 1A and 1B, the portion of the inner tubular member 200 which is inside the outer tubular member 300 is illustrated with darker dotted lines, and the portion of the suction member 100 that is inside the inner tubular member 200 is illustrated with lighter dotted lines.

The suction member 100 includes a handle portion 102, a first connecting portion 104, an extended portion 106 (sometimes also referred to as an extended central portion), a second connecting portion 108, and an end portion 110 (see FIG. 2A). The first connecting portion 104 connects the handle portion 102 to the extended portion 106 and the second connecting portion 108 connects the extended portion 106 to the end portion 110. The extended portion 106 may include a sealing portion 116 (e.g., fabricated from rubber) and the end portion 110 includes a side surface 112 (e.g., side surface 112 corresponding to a side surface of a cylinder or a side surface of a cone) and a top surface 114 (e.g., a tip that is rounded).

The inner tubular member 200 includes a first portion 202, a second portion 204, a first end 206, a second end 208, a first opening 210 at (or near) the first end 206, a second opening 212 at (or near) the second end 208, an inner surface 214, an outer surface 216, a protrusion 218 (e.g., a button shaped protrusion, linear protrusion, or a diamond-shaped protrusion), and a handle 220 (see FIG. 2B). The first portion 202 of the inner tubular member 200 may be a first tubular portion including the first end 206 and the first opening 210 at the first end 206. The second portion 204 of the inner tubular member 200 may be a second tubular portion (e.g., a cylindrical portion) including the second end 208 and the second opening 212 at the second end 208. The inner surface 214 of the first portion 202 may correspond to a cylindrical shape (or an elliptical cylinder shape), although the outer surface 216 of the first portion 202 may not be exactly cylindrical. However, it should be understood that in some embodiments, the outer surface 216 of the first portion 202 may be cylindrical (or an elliptical cylinder shape). The protrusion 218 may be formed on the outer surface 216 and may correspond to a button shaped protrusion that extends away from the inner tubular member 200 (e.g., extends away from the outer surface 216 of the second portion 204 of the inner tubular member 200). In other words, the protrusion 218 extends away from an axis formed by center points of the first opening 210 and the second opening 212 or an axis formed by a length of the suction member 100 in the inner tubular member 200. In some embodiments, the protrusion 218 may correspond to any bulge, projection, or a jut formed on the outer surface 216 and that extends away from the from the inner tubular member 200 (e.g., extends away from the outer surface 216 of the second portion 204). In some embodiments, the protrusion 218 may be formed by any other shape (e.g., cylindrical, semi spherical, conical, linear, diamond). Although the protrusion 218 has been illustrated as being formed on the outer surface 216 of the second portion 204 of the inner tubular member 200, it should be understood that in some embodiments, the protrusion 218 may be formed anywhere on the outer surface 216 of the inner tubular member 200, including on the outer surface 216 of the first portion 202 of the inner tubular member 200. The handle 220 may be formed close to (or at) the second end 208 and handle 220 extends away from the inner tubular member 200 (e.g., extends away from the outer surface 216 of the second portion 204 of the inner tubular member 200 or extends away from the second end 208 of the inner tubular member 200). The inner tubular member 200 may include one or more handles 220. In some embodiments, the handle 220 may be formed at the second end 208 and extends away from the inner tubular member 200 (e.g., extends away from the outer surface 216 of the second portion 204 of the inner tubular member 200). Although a single protrusion is illustrated in the figures, it should be understood that there may be a plurality of protrusions and such one or more protrusions may be formed on the outer tubular member 300 (in addition to or alternative to forming on the inner tubular member 200).

The outer tubular member 300 includes a first portion 302, a second portion 304, a first end 306, a second end 308, a first opening 310 at (or near) the first end 306, a second opening 312 at (or near) the second end 308, an inner surface 314, an outer surface 316, a notch 318, and a tab 320 (see FIG. 2C). The first portion 302 of the outer tubular member 300 may be a first tubular portion including the first end 306 and the first opening 310 at the first end 306. The second portion 304 of the outer tubular member 300 may be a second tubular portion (e.g., a cylindrical portion) including the second end 308 and the second opening 312 on the second end 308. The outer tubular member 300 may have the same or similar inner configuration (e.g., shape) as the inner tubular member 200. In other words, the first portion 302 of the outer tubular member 300 may have the same or similar configuration (e.g., shape) as the first portion 202 of the inner tubular member 200 and the second portion 304 of the outer tubular member 300 may have the same or similar configuration as the second portion 204 of the inner tubular member 200. In some embodiments, the inner surface 314 of the outer tubular member 300 may have the same configuration as the outer surface 216 of the inner tubular member 200. In some embodiments, the outer tubular member 300 may include a notch 318. The notch 318 (e.g., a cutout) may include a first portion 322 that is formed parallel to an axis formed by a length of the suction member 100 when the suction member 100 is in the inner tubular member or an axis formed by the center points of the first opening 210 and second opening 212 of the inner tubular member 200. The second portion 324 may be formed at a 90-degree angle (e.g., perpendicular or substantially perpendicular) from the first portion 322. However, since the second portion 304 of the outer tubular member 300 may be cylindrically shaped, the second portion 324 of the notch 318 may be formed corresponding to the curvature of the outer tubular member 300. Although the notch 318 is illustrated as being formed on the second portion 304 of the outer tubular member 300, it should be understood that the notch 318 may be formed anywhere on the outer tubular member 300, including being formed on the first portion 302 of the outer tubular member 300. The first portion 322 of the notch 318 and the second portion 324 of the notch 318 may form an “L” shape on the outer surface 316 of the outer tubular member 300. Although FIG. 2C illustrates a certain configuration of the second portion 324 relative to the first portion 322, it should be understood that the second portion 324 may be formed on either side of the first portion 322 to create the “L” shape.

Further, in some embodiments, the notch 318 may correspond to different shapes other than the “L” shape illustrated in the figures. The notch 318 may further include a groove 326 at the second end 308 of the outer tubular member 300 (see FIG. 2F). Having a groove 326 instead of a cut at the second end 308 of the outer tubular member 300 ensures structural stability of the outer tubular member 300. The second end 308 may include a ring-shaped portion and the tab 320 may extend away from such a ring-shaped portion. Further, the tab 320 may be formed close to the second end 308 and extends away from the outer tubular member 300 (e.g., extends away from the outer surface 316 of the second portion 304). Although one tab 320 is illustrated in FIGS. 1A, 1B, 2C, 2F, and 3A-3F, it should be understood that the outer tubular member 300 may include one or more tabs 320. In some embodiments, the notch 318 may be formed adjacent to the tab 320, as illustrated in FIG. 2C.

In some embodiments, there may be a plurality of notches 318 or the notch 318 may correspond to a different shape. For example, the notch 318 may be J-shaped, T-shaped, or U shaped. Having a J-shaped notch (e.g., having a curved first portion 322) may facilitate smoother disengagement of the outer tubular member 300 from the inner tubular member 200. Further, for example, a U-shaped notch may include a plurality of first portions 322, thereby allowing entry and exit of the protrusion 218 via different first portions 322 of the notch 318. In some embodiments, the second portion 324 of the notch 318 may span the entire curvature of the outer tubular member 300. In addition to the notch 318 spanning an entire curvature of the outer tubular member 300, in some embodiments, there may be a plurality of first portions 322 connected to the second portion 324, thereby allowing entry and exit of the protrusion via a plurality of first portions 322. In some embodiments, having a plurality of first portions 322 may be advantageous because it may allow both a right-handed person and a left-handed person to conveniently lock and unlock the ligation device 10 while using such a device for treating a patient. Having a T-shaped notch is also advantageous as it allows the ligation device 10 to be locked and unlocked by rotating the outer tubular member 300 (or the inner tubular member 200) in both a clockwise and a counterclockwise direction.

As illustrated in FIG. 2G, in some embodiments, the outer tubular member 300 may only include the first portion 322 of the notch 318. In such a case, the first portion 322 may be narrow in some portions (e.g., portion 328 of the first portion 322) and wider in other portions (e.g., portions 330 of the first portion 322). In other words, the width of the portion 322 may not be even throughout a length of the first portion 322. For example, a portion (e.g., portion 328) of the first portion 322 may be narrower than other portions (e.g., portion 330) of the first portion 322, and the other portions (e.g., portion 330) of the first portion 322 may be wider than the narrower portion (e.g., portion 328) of the first portion 322. When a protrusion 218 is received within such a first portion 322 of the notch 318, the contact between the narrower portion (e.g., portion 328) of the first portion 322 and the protrusion 218 may prevent accidental or unintended movement of the outer tubular member 300 towards a first end 206 of the inner tubular member 200, thereby preventing early deployment of an elastic band. The width of the narrower portion (e.g., portion 328) of the first portion 322 may be similar or substantially similar to the width of the protrusion 218. It should be understood that the narrower portion 328 may be formed anywhere within the first portion 322. An advantage of such a configuration (as illustrated in FIG. 2G) is that a physician (or any professional) that is using the ligation device 10 may not need to twist the outer tubular member 300 or the inner tubular member 200 to lock and unlock the outer tubular member 300 and the inner tubular member 200.

Further, the ligation device 10 includes a stopper member 400, which includes a first portion 402, a second portion 404, and an opening 406 (see FIG. 2D). The first portion 402, second portion 404, and opening 406 may correspond to a shape of the second opening 212 of the inner tubular member 200. The opening 406 of the stopper member 400 may be smaller than the second opening 212 of the inner tubular member 200 and the opening 406 of the stopper member 400 and the second opening 212 of the inner tubular member may be concentric. The ligation device 10 also includes a loading member 500, which includes a first end 502 and a second end 504 (see FIG. 2E). The second end 504 may correspond to a shape of the first opening 210 of the inner tubular member 200 and may slidably fit into the first opening 210 of the inner tubular member 200 to help with the loading of an elastic band on (or near) the first end 206 of the inner tubular member 200 and adjacent to the first end 306 of the outer tubular member 300.

Although the separate components of the ligation device 10 are described above, the following description provides details regarding the relationship between the different components of the ligation device 10.

The suction member 100 is at least partially within the inner tubular member 200 (see FIGS. 1A and 1B). The handle portion 102 of the suction member 100 may be used to pull the suction member 100 away from the first end 206 of the inner tubular member 200 and may be used to push the suction member 100 towards the first end 206 of the inner tubular member 200 (or the handle portion 102 of the suction member 100 may be used to pull the suction member 100 towards the second end 208 of the inner tubular member 200 and may be used to push the suction member 100 away from the second end 208 of the inner tubular member 200). When the suction member 100 is pushed toward the first end 206 of the inner tubular member 200, the end portion 110 of the suction member 100 may be exposed such that the end portion 110 may either partially or fully cross a plane formed by the circumference of the first opening 210 of the inner tubular member 200 (see FIGS. 1A and 1B). The extended portion 106 that slidably engages (e.g., hugs) with the inner surface 214 of the second portion 204 of the inner tubular member 200 prevents the extended portion 106 from being pushed into the first portion 202 of the inner tubular member 200, thereby preventing the suction member 100 from being pushed beyond a certain point (see FIGS. 1A, 1B, and 2A). When the suction member 100 is pulled towards the second end 208 of the inner tubular member 200, the end portion 110 of the may be pulled inside the inner tubular member 200 such that the end portion 110 may either partially or fully cross a plane formed by the circumference of the first opening 210 of the inner tubular member 200.

In some embodiments, the stopper member 400 that is disposed at (or attached to) the second end 208 of the inner tubular member 200 prevents the suction member 100 from being entirely pulled out of the inner tubular member 200 (see FIGS. 2D and 4B). This is advantageous because when a physician (or any professional) performs a procedure on a patient using a ligation device, the physician may accidently pull the suction mechanism forcefully, which may not only result in the such suction mechanism detaching from a ligation device, but may also injure a patient. Specifically, the force of detaching the suction mechanism from an inner tubular member may cause other components of a ligation device to be forced to move into a patient's rectum during hemorrhoidal treatment of a patient using the ligation device, which can be dangerous for the patient if the ligation device is already inside a person's rectum. Such movement of the ligation device can cause injury to the person's sensitive tissue inside the person's rectum. The stopper member 400 prevents such detachment of the suction member 100 from the inner tubular member 200 and may prevent injury to a patient that receives treatment using the ligation device 10. The stopper member 400 includes a first portion 402 that attaches to the second end 208 (or a portion of the handle 220) of the inner tubular member 200 and the second portion 404 attaches to the inner surface 214 of the second portion 204 of the inner tubular member 200 that is adjacent to the second opening 212 of the inner tubular member 200 (see FIGS. 2D and 4B). The first portion 402 and the second portion 404 of the stopper member 400 may be attached to the inner tubular member 200 using a strong adhesive to prevent the suction member 100 from being entirely pulled out of the inner tubular member 200. In some embodiments, the stopper member 400 may be attached to the inner tubular member 200 using sonic welding or ultrasonic welding techniques. The second portion 404 of the stopper member 400 may prevent the suction member 100 from being entirely pulled out of the inner tubular member 200 (see FIGS. 2D and 4B). Specifically, the stopper member 400 acts as a blocker to prevent the suction member 100 from completely detaching from the inner tubular member 200.

The stopper member 400 also includes an opening 406 that allows the first connecting portion 104 of the suction member 100 to travel in and out of the inner tubular member 200 (see FIGS. 2D and 4B). Specifically, when the suction member 100 is pulled away from the first end 206 of the inner tubular member 200, the first connecting portion 104 of the suction member 100 travels out of the inner tubular member 200 and when the suction member 100 is pushed toward the first end 206 of the inner tubular member 200, the first connecting portion 104 of the suction member 100 travels into the inner tubular member 200. The width (or diameter) of the opening 406 of the stopper member 400 is less than the width of the extended portion 106 (including the width of the sealing portion 116). Since the opening 406 of the stopper member 400 is less than the width of the extended portion, the suction member 100 is prevented from being completely detached from the inner tubular member 200 via the opening 406 of the stopper member 400. The opening 406 of the stopper member 400 and the second opening 212 of the inner tubular member 200 are concentric. In some embodiments, the configuration of the opening 406 of the stopper member 400 may correspond to a shape of the first connecting portion 104 of the suction member 100.

In some embodiments, the inner tubular member 200 includes a protrusion 218 (see FIGS. 1A, 1B, 2B, 3A, 3C, 3E, 4A, and 4B). The protrusion may be formed on the outer surface 216 of the inner tubular member 200. Further, in some embodiments, the outer tubular member 300 includes a notch 318 that is configured to receive the protrusion 218 of the inner tubular member 200. The size (e.g., width, length, diameter) of the protrusion 218 may be smaller than the width of the first portion 322 of the notch 318 and the width of the second portion 324 of the notch 318. The width of the first portion 322 of the notch 318 may be substantially similar to the width of the second portion 324 of the notch 318. In some embodiments, the width of the second portion 324 of the notch 318 may be smaller than the width of the first portion 322 of the notch 318. The size (e.g., height of the protrusion that extends away from the outer surface 216 of the inner tubular member 200) may be larger than the thickness (e.g., thickness measured perpendicular to an axis formed by the length of the suction member 100 in the inner tubular member 200 or an axis formed by the center points of the first opening 210 and the second opening 212 of the inner tubular member 200) of the outer tubular member 300 or the thickness of the notch 318 formed on the outer tubular member 300. The inner configuration of the outer tubular member 300 may correspond to an outer configuration of the inner tubular member 200. In other words, the configuration of the inner surface 314 of the outer tubular member 300 may correspond to the configuration of the outer surface 216 of the inner tubular member 200. When the outer tubular member 300 is placed over the inner tubular member 200, the outer tubular member 300 may be placed such that the notch 318 (e.g., first portion 322 of the notch 318 illustrated in FIGS. 1B and 3E) of the outer tubular member 300 receives the protrusion 218 of the inner tubular member 200. As illustrated in FIGS. 1B and 3E, the outer tubular member 300 may be placed over the inner tubular member 200 such that the protrusion 218 of the inner tubular member 200 is received by the first portion 322 of the notch 318 and the protrusion 218 slides along a length of the first portion 322 of the notch 318 of the outer tubular member 300. This position (e.g., the position of protrusion 218 along the length of the first portion 322 of the notch 318 illustrated in FIGS. 1B and 3E) corresponds to an unlocked position because the outer tubular member 300 can be removed (or moved towards the first end 206 of the inner tubular member 200) when the protrusion 218 is within any portion of the first portion 322 of the notch 318. FIGS. 1B, 3E, and 3F illustrate such an unlocked position.

In some embodiments, the inner tubular member 200 includes a handle 220 and the outer tubular member 300 includes a tab 320. The handle 220 and the tab 320 allow a person to properly grip the ligation device 10 while treating a patient for hemorrhoids using such a ligation device 10 and the tab 320 helps with the release of an elastic band. As illustrated in FIGS. 1B, 2B, and 3E, the handle 220 may extend away from the second end 208 of the inner tubular member 200 or from the outer surface 216 of the inner tubular member 200 (e.g., away from an axis formed by the center points of the first opening 210 and the second opening 212 of the inner tubular member 200). An axis formed by such an extension of the handle 220 is illustrated in FIG. 3F as a solid arrow. FIG. 3F corresponds to a different view (showing a view of the inner tubular member 200 and the outer tubular member 300 from a bottom view corresponding to the second opening 212 of the inner tubular member 200) of the configuration illustrated in FIG. 3E. The axis (see solid arrow in FIG. 3F) formed by the handle 220 may correspond to an axis extending away from an axis formed by the center points of the first opening 210 and the second opening 212 of the inner tubular member 200. Further, as illustrated in FIGS. 1B, 2B, and 3E, the tab 320 may extend away from the second end 308 of the outer tubular member 300 or from the outer surface 316 of the outer tubular member 300 (e.g., away from an axis (not shown) formed by the center points of the first opening 210 and the second opening 212 of the inner tubular member 200). An axis formed by such an extension of the tab 320 is illustrated in FIG. 3F as a dotted arrow. The axis (see dotted arrow in FIG. 3F) formed by the tab 320 may correspond to an axis extending away from an axis formed by the center points of the first opening 210 and the second opening 212 of the inner tubular member 200. An angle between the axis formed by the tab 320 and the axis formed by the handle 220 (from a view into the inner tubular member 200 via the second opening 212 in FIG. 3F) may correspond to an angle of about 0 to 45 degrees. For example, forming an angle of about 45 degrees between the solid arrow and the dotted arrow in FIG. 3F may correspond to an unlocked position (see FIG. 3E). Rotating the outer tubular member 300 (or the inner tubular member 200) by reducing the angle between the dotted and the solid arrow will lock the outer tubular member 300 and the inner tubular member 200 (because the protrusion 218 will move into the second portion 324 of the notch 318) and prevent the outer tubular member 300 from being removed or from being moved towards the first end 206 of the inner tubular member 200. Details regarding the locked position is described below.

By twisting (e.g., either clockwise or counterclockwise depending on whether the second portion 324 of the notch 318 is in a clockwise or counterclockwise direction relative to the first portion 322 of the notch 318) the outer tubular member 300 (or the inner tubular member 200 or both) such that the protrusion 218 is received by the second portion 324 of the notch 318, the outer tubular member 300 and the inner tubular member 200 are locked such that the outer tubular member 300 cannot be moved towards the first end 206 of the inner tubular member 200 (or moved away from the first end 206 the inner tubular member 200), thereby preventing accidental or unintended movements of the outer tubular member 300 relative to the inner tubular member 200. This position (e.g., the position of protrusion 218 along the length of the second portion 324 of the notch 318, as illustrated in FIGS. 1A, 3A, and 3C) corresponds to a locked position because the outer tubular member 300 cannot be moved towards the first end 206 of the inner tubular member 200 when the protrusion 218 is within any portion of the second portion 324 of the notch 318. FIGS. 1A, 3A, and 3C illustrate such a locked position. To unlock, the outer tubular member 300 (or the inner tubular member 200) may be twisted (e.g., either clockwise or counterclockwise depending on whether the first portion 322 of the notch 318 is in a clockwise or counterclockwise direction relative to the second portion 324 of the notch 318) such that the protrusion 218 is within the first portion 322 of the notch 318. When the protrusion 218 is within the first portion 322 of the notch 318, the outer tubular member 300 can be moved towards the first end 206 of the inner tubular member, thereby allowing the outer tubular member 300 to be detached from the inner tubular member 200.

As noted above, in some embodiments, the inner tubular member 200 includes a handle 220 and the outer tubular member 300 includes a tab 320. The handle 220 and the tab 320 allow a person to properly grip the ligation device 10 while treating a patient for hemorrhoids using such a ligation device 10 and the tab 320 helps with the release of an elastic band. As illustrated in FIGS. 1A, 2B, 3A, and 3C, the handle 220 may extend away from the second end 208 of the inner tubular member 200 or from the outer surface 216 of the inner tubular member 200 (e.g., away from an axis formed by the center points of the first opening 210 and the second opening 212 of the inner tubular member 200). An axis formed by such an extension of the handle 220 is illustrated in FIGS. 3B and 3D as a solid arrow. FIGS. 3B and 3D correspond to different views (showing views of the inner tubular member 200 and the outer tubular member 300 from a bottom view corresponding to the second opening 212 of the inner tubular member 200) of the configurations illustrated in FIGS. 3A and 3C, respectively. The axis (see solid arrows in FIGS. 3B and 3D) formed by the handle 220 may correspond to an axis extending away from an axis formed by the center points of the first opening 210 and the second opening 212 of the inner tubular member 200. Further, as illustrated in FIGS. 1A, 2B, 3A, and 3C, the tab 320 may extend away from the second end 308 of the outer tubular member 300 or from the outer surface 316 of the outer tubular member 300 (e.g., away from an axis (not shown) formed by the center points of the first opening 210 and the second opening 212 of the inner tubular member 200). An axis formed by such an extension of the tab 320 is illustrated in FIGS. 3B and 3D as a dotted arrow. The axis (see dotted arrow in FIGS. 3B and 3D) formed by the tab 320 may correspond to an axis extending away from an axis formed by the center points of the first opening 210 and the second opening 212 of the inner tubular member 200. An angle between the axis formed by the tab 320 and the axis formed by the handle 220 (from a view into the inner tubular member 200 via the second opening 212 in FIGS. 3B and 3D) may correspond to an angle of about 0 to 45 degrees.

As illustrated in FIG. 3B, an angle of about 45 degrees between the solid arrow and the dotted arrow corresponds to a locked position (see also FIG. 3A). Further, as illustrated in FIG. 3D, an angle of about 0 degrees between the solid arrow and the dotted arrow also corresponds to a locked position (see also FIG. 3C). Having the angle as 0 degrees in a locked position is advantageous when the ligation device 10 is inserted into a patient's rectum. It helps a physician properly grip the ligation device 10 (using the handle 220 and the tab 320) and prevents unintended or accidental movement of the outer tubular member 300 towards the first end 206 of the inner tubular member.

As noted above, the locking mechanism described above ensures that the outer tubular member 300 does not move (e.g., in a direction corresponding to an axis formed by the length of the suction member or an axis formed by the center points of the first opening 210 and the second opening 212 of the inner tubular member 200) relative to the inner tubular member 200. Of course, the outer tubular member 300 (or the inner tubular member 200) may be rotated relative to the inner tubular member 200 (or the outer tubular member 300) in a locked position in order to unlock the outer tubular member 300 from the inner tubular member 200.

The locking mechanism described above is advantageous because it prevents any vertical movement (e.g., in a direction corresponding to an axis formed by the length of the suction member or an axis formed by the center points of the first opening 210 and the second opening 212 of the inner tubular member 200) of the outer tubular member 300 relative to the inner tubular member 200 (e.g., prevents movement of the outer tubular member 300 towards the first end 206 of the inner tubular member 200). Unintended or accidental movement of the outer tubular member 300 (e.g., toward the first end 206 of the inner tubular member) during treatment of hemorrhoids can cause premature deployment of an elastic band that is attached over the first end 206 of the inner tubular member 200 and that is adjacent to the first end 306 of the outer tubular member 300 (the elastic band may be attached to the first end 206 of the inner tubular member 200 using the loading member 500. For example, the second end 504 of the loading member can be inserted into the first opening 210 of the inner tubular member 200 and an elastic band can be attached to the first end 206 of the inner tubular member 200 via the first end 502 of the loading member 500). Not only can this lead to added costs and time to perform a procedure on a patient, unintended vertical movement of the outer tubular member 300 relative to the inner tubular member 200 (e.g., unintended movement of the outer tubular member 300 towards the first end 206 of the inner tubular member 200) can also cause injury to the sensitive human tissue in the rectum of a human while treatment is being performed using a ligation device.

Although U.S. Pat. No. 9,622,750 attempted to solve the premature deployment of the elastic band using a locking ring formed on an inner tubular member and lug extending transversely from a tab towards the inner tubular member, U.S. Pat. No. 9,622,750 fails to prevent unintended movement of the outer tubular member towards the front end of the inner tubular member. In other words, a small amount of force may be sufficient to cause the unintended movement of the outer tubular member in U.S. Pat. No. 9,622,750 towards the front end of the inner tubular member, which can result in premature deployment of the elastic band and injuries to a patient that is receiving treatment using a ligation device of U.S. Pat. No. 9,622,750. The present embodiments solve this problem by preventing unintended movement of the outer tubular member 300 towards the first end 206 of the inner tubular member 200, thereby providing added safety for a patient receiving treatment using the ligation device 10 in addition to reducing cost and saving time associated with premature deployment of the elastic band. Also, as noted above, allowing the angle between an axis formed by the tab 320 and the axis formed by the handle 220 to be about 0 degrees in a locked position helps physician properly grip the ligation device 10 (using the handle 220 and the tab 320) and prevents unintended or accidental movement of the outer tubular member 300 towards the first end 206 of the inner tubular member.

It should be understood that the notch 318 and the protrusion 218 may be formed anywhere on the outer tubular member 300 and the inner tubular member 200, respectively, that allows the angle between the solid arrow and the dotted arrow to be 0 degrees in a locked position. In some embodiments, the notch 318 may be formed on the inner tubular member 200 and an inward protrusion may be formed on the inner surface 314 of the outer tubular member 300. Such an inward protrusion may extend towards an axis formed by the center points of the first opening 210 and the second opening 212 of the inner tubular member 200 (or an axis formed by the center points of the first opening 310 and the second opening 312 of the outer tubular member 300). Such an inward protrusion on the inner surface 314 of the outer tubular member 300 and the notch 318 on the inner tubular member 200 may be used as a locking mechanism.

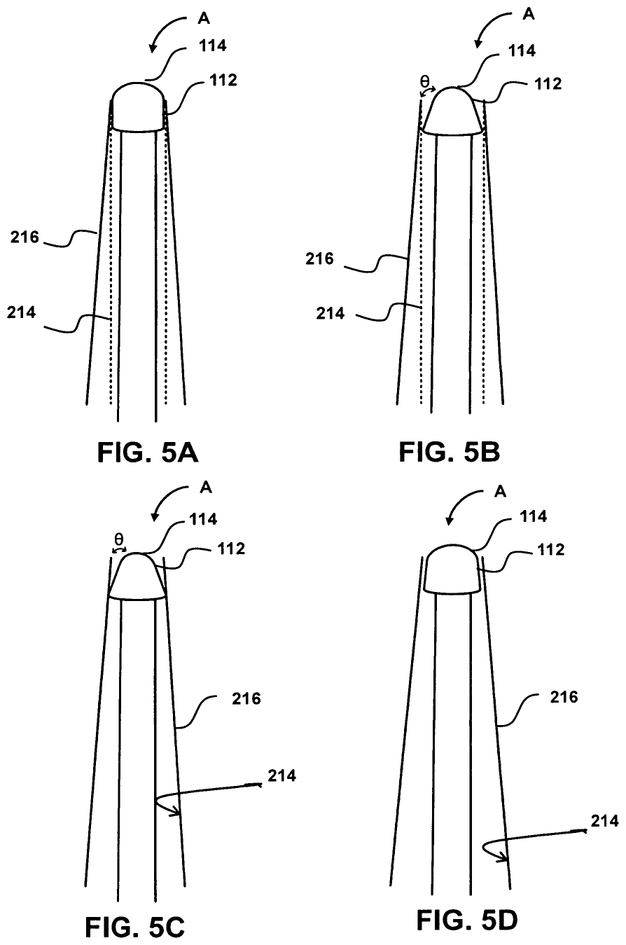

In some embodiments, the end portion 110 of the suction member 100 may have different configurations relative to the inner tubular member 200 (e.g., relative to the inner surface 214 of the inner tubular member). FIG. 4A illustrates portion A (which corresponds to the suction member 100 and the inner tubular member 200), and FIGS. 5A-D illustrates the different configurations of this portion A. As illustrated in FIG. 5A, the side surface 112 of the end portion 110 may be parallel (or substantially parallel) to the inner surface 214 of the inner tubular member 200. Although an outer surface 216 of the inner tubular member may not be cylindrical, the inner surface 214 of portion A may be cylindrical (or an elliptical cylinder shape) such that the inner surface 214 of the inner tubular member is parallel (or substantially parallel) to the side surface 112 of the end portion 110 of the suction member 100. As illustrated in FIG. 5A, the side surface 112 may correspond to a side surface of a cylinder and the top surface 114 of the end portion 110 may be rounded. Having the side surface 112 of the end portion 110 be parallel (or substantially parallel) to the inner surface 214 of the inner tubular member 200 improves the stability of the ligation device 10 when drawing human tissue (e.g., rectal or hemorrhoidal tissue) into the inner tubular member 200.

In FIG. 5B, the side surface 112 of the end portion 110 of the suction member 100 and the inner surface 214 of the inner tubular member 200 may form an angle theta (θ). This angle may be greater than approximately 30 degrees. In FIG. 5C, the side surface 112 of the end portion 110 of the suction member 100 and the inner surface 214 of the inner tubular member 200 may form an angle theta (θ). This angle may be greater than approximately 30 degrees. In FIG. 5D, the side surface 112 of the end portion 110 of the suction member 100 and the inner surface 214 of the inner tubular member 200 may be parallel (or substantially parallel). In FIGS. 5B-5D, the side surface 112 of the end portion 110 of the suction member 100 converges towards the top surface 114 (e.g., a tip that is rounded) of the end portion 110 of the suction member 100. By having the suction member 100 converge towards the top surface 114, pinching of human tissue may be reduced when the suction member 100 is retracted into the inner tubular member 200 while inside a patient's rectum. FIGS. 5A and 5B illustrate a spacing between the inner surface 214 and the outer surface 216 of the inner tubular member 200. Such spacing may include the same (or similar) material as the material forming the inner surface 214 or the outer surface 216 of the inner tubular member 200. In some embodiments, such spacing may be an airtight space between the inner surface 214 and the outer surface 216 of the inner tubular member 200.

Further, in some embodiments, because of the stopper member 400, the suction member 100 may only be pulled towards the second end 208 of the inner tubular member until the stopper member 400 contacts the extended portion 106 of the suction member 100. At this point (e.g., where the stopper member 400 contacts the extended portion 106 of the inner tubular member 100), the side surface 112 may continue to form the same angles illustrated in FIGS. 5A-5D. For example, at this point (e.g., where the stopper member 400 contacts the extended portion 106 of the inner tubular member 100), the side surface 112 of the end portion 110 of the suction member 100 may continue to be parallel (or substantially parallel) to the inner surface 214 of the inner tubular member 200. As such, regardless of any pull or push of the suction member 100, the angle formed between the side surface 112 of the end portion 110 of the suction member 100 and the inner surface 214 of the inner tubular member 200 may remain the same at any point in the ligation device 10.

The ligation device 10 may be fabricated from plastic and the sealing portion 116 of the suction member can be fabricated from rubber. In some embodiments, the ligation device 10 may be fabricated using metal. In some embodiments, the ligation device 10 may include visual guides for using the ligation device 10 (e.g., using the locking mechanism of the ligation device 10). The visual guides may be color coded or may include markings on the ligation device 10. In some embodiments, the shape of the handle 220 and the tab 320 may not limited to the shapes illustrated in the figures. For example, the handle 220 and the tab 320 may be ring-shaped or any other shape. In some embodiments, the outer tubular member 300 and the inner tubular member 200 may include a clicking mechanism that allows the outer tubular member 300 and the inner tubular member 200 to be locked via a clicking mechanism.

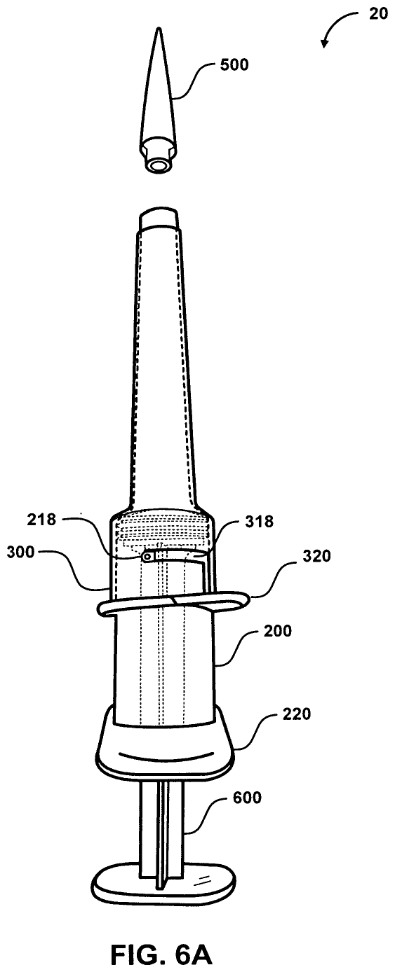

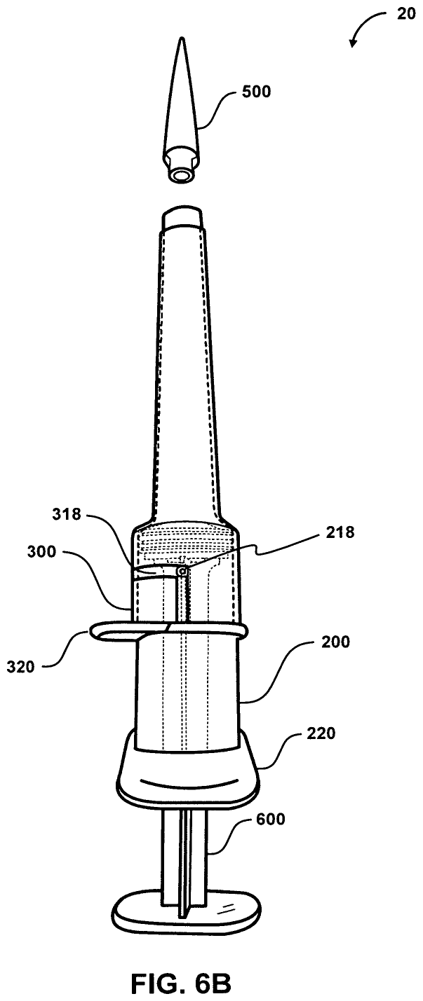

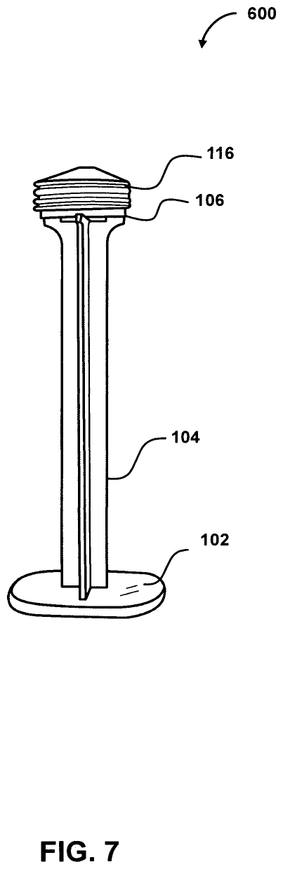

FIGS. 6A, 6B, and 7 illustrate another embodiment of the ligation device 20. FIG. 6A is similar to FIG. 1A except that FIG. 6A does not include the second connecting portion 108 and the end portion 110 of the suction member 100. FIG. 6B is similar to FIG. 1B except that FIG. 6B does not include the second connecting portion 108 and the end portion 110 of the suction member 100. FIG. 7 is similar to FIG. 2A except that FIG. 7 does not include the second connecting portion 108 and the end portion 110 of the suction member 100. The suction member 100 without the second connecting portion 108 and the end portion 110 is illustrated as suction member 600 in FIGS. 6A, 6B, and 7. All of the description above applies to FIGS. 6A, 6B, and 7, and therefore, for the sake of brevity, the details are not repeated.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The present techniques will be better understood with reference to the following enumerated embodiments:
1. A device, comprising: an inner tubular member having an inner surface, an outer surface, a first end, a second end, and a protrusion on the outer surface, the first end having a first opening and the second end having a second opening; and an outer tubular member having an inner configuration corresponding to that of the inner tubular member, the outer tubular member including a notch that is configured to receive the protrusion of the inner tubular member, wherein a reception of the protrusion of the inner tubular member by a first portion of the notch of the outer tubular member allows movement of the outer tubular member toward the first end of the inner tubular member and a reception of the protrusion of the inner tubular member by a second portion of the notch of the outer tubular member prevents the movement of the outer tubular member toward the first end of the inner tubular member.
2. The device of embodiment 1, wherein the device is a ligation device for treating hemorrhoids including a locking mechanism.
3. The device according to any of embodiments 1-2, further comprising: a suction member partially in the inner tubular member, the suction member including a handle portion and an end portion, and the handle portion allowing the end portion of the suction member to be pulled away from the first end of the inner tubular member.
4. The device according to any of embodiments 1-3, further comprising: a stopper member attached to the second end of the inner tubular member, the stopper member preventing the suction member from being entirely pulled out of the inner tubular member.
5. The device according to any of embodiments 1-4, wherein the outer tubular member includes a handle, the handle forming a second axis extending away from a first axis formed by center points of the first opening and the second opening of the inner tubular member, and the inner tubular member includes a tab, the tab forming a third axis extending away from the first axis formed by the center points of the first opening and the second opening of the inner tubular member.
6. The device according to any of embodiments 1-5, wherein the second axis and the third axis create an angle of about 0-45 degrees.
7. The device according to any of embodiments 1-6, wherein the reception of the protrusion of the inner tubular member by the second portion of the notch of the outer tubular member allows the outer tubular member to be rotated relative to the inner tubular member about a first axis formed by center points of the first opening and the second opening of the inner tubular member.
8. The device of any of embodiments 1-7, wherein the reception of the protrusion of the inner tubular member by the second portion of the notch of the outer tubular member allows the inner tubular member to be rotated relative to the outer tubular member about a first axis formed by center points of the first opening and the second opening of the inner tubular member.
9. The device of any of embodiments 1-8, wherein the protrusion on the outer surface of the inner tubular member is a button shaped protrusion.
10. The device of any of embodiments 1-9, wherein a length of the protrusion extending away from the inner tubular member is greater than a thickness of the notch formed on the outer tubular member.
11. The device of any of embodiments 1-10, wherein the first portion of the notch is parallel to a first axis formed by center points of the first opening and the second opening of the inner tubular member, and the second portion of the notch is perpendicular to the first portion.
12. The device of any of embodiments 1-11, wherein the first portion of the notch and the second portion of the notch form an L-shape on the outer tubular member.
13. The device of any of embodiments 1-12, wherein the end portion of the suction member includes a side surface and a top surface.
14. The device of any of embodiments 1-13, wherein the side surface of the end portion of the suction member is substantially parallel to the inner surface of the inner tubular member that is adjacent to the first opening of the inner tubular member.
15. The device of any of embodiments 1-14, wherein the side surface of the end portion of the suction member and the inner surface of the inner tubular member that is adjacent to the first opening of the inner tubular member form an angle of about 30 degrees or more.
16. The device of any of embodiments 1-15, wherein the stopper member has a shape conforming to a shape of the second opening of the inner tubular member, the stopper member includes an opening, and the suction member is pulled away from the first end of the inner tubular member and pushed toward the first end of the inner tubular member via the opening of the stopper member.
17. The device of any of embodiments 1-16, wherein the stopper member includes a first portion and a second portion.
18. The device of any of embodiments 1-17, wherein the first portion of the stopper member contacts the second end of the inner tubular member.
19. The device of any of embodiments 1-18, wherein the second portion of the stopper member contacts a portion of the inner surface of the inner tubular member that is adjacent to the second opening of the inner tubular member such that the portion of the inner surface of the inner tubular member hugs the second portion of the stopper member.
20. The device of any of embodiments 1-19, wherein the opening of the stopper member and the second opening of the inner tubular member are concentric.
21. The device of any of embodiments 1-20, wherein the suction member further includes an extended central portion and a connecting portion, the connecting portion connecting the extended central portion to the handle portion, and a width of the extended central portion is larger than a diameter of an opening of the stopper member to prevent the suction member from being entirely pulled out of the inner tubular member.
22. The device of any of embodiments 1-21, wherein the end portion of the suction member includes a side surface and a top surface, and the side surface is parallel to a portion of the inner surface of the inner tubular member that is adjacent to the first opening of the inner tubular member.
23. The device of any of embodiments 1-22, wherein the side surface of the end portion of the suction member is cylindrical in shape, and the top surface of the end portion of the suction member is rounded.
24. A device, comprising: an inner tubular member having an inner surface, an outer surface, a first end, a second end, and a protrusion on the outer surface, the first end having a first opening and the second end having a second opening; and an outer tubular member having an inner configuration corresponding to that of the inner tubular member, the outer tubular member including a notch that is configured to receive the protrusion of the inner tubular member, the notch including a wider portion and a narrower portion, the narrower portion of the notch preventing movement of the outer tubular member toward the first end of the inner tubular member.
25. The device of embodiment 24, wherein the device is a ligation device for treating hemorrhoids including a locking mechanism.
26. The device according to any of embodiments 24-25, further comprising: a suction member partially in the inner tubular member, the suction member including a handle portion and an end portion, and the handle portion allowing the end portion of the suction member to be pulled away from the first end of the inner tubular member.
27. The device according to any of embodiments 24-26, further comprising: a stopper member attached to the second end of the inner tubular member, the stopper member preventing the suction member from being entirely pulled out of the inner tubular member.
28. The device according to any of embodiments 24-27, wherein the outer tubular member includes a handle, the handle forming a second axis extending away from a first axis formed by center points of the first opening and the second opening of the inner tubular member, and the inner tubular member includes a tab, the tab forming a third axis extending away from the first axis formed by the center points of the first opening and the second opening of the inner tubular member.
29. The device according to any of embodiments 24-28, wherein the second axis and the third axis create an angle of about 0-45 degrees.
30. The device of any of embodiments 24-29, wherein the protrusion on the outer surface of the inner tubular member is a button shaped protrusion.
31. The device of any of embodiments 24-30, wherein a length of the protrusion extending away from the inner tubular member is greater than a thickness of the notch formed on the outer tubular member.
32. The device of any of embodiments 24-31, wherein the end portion of the suction member includes a side surface and a top surface.
33. The device of any of embodiments 24-32, wherein the side surface of the end portion of the suction member is substantially parallel to the inner surface of the inner tubular member that is adjacent to the first opening of the inner tubular member.
34. The device of any of embodiments 24-33, wherein the side surface of the end portion of the suction member and the inner surface of the inner tubular member that is adjacent to the first opening of the inner tubular member form an angle of about 30 degrees or more.
35. The device of any of embodiments 24-34, wherein the stopper member has a shape conforming to a shape of the second opening of the inner tubular member, the stopper member includes an opening, and the suction member is pulled away from the first end of the inner tubular member and pushed toward the first end of the inner tubular member via the opening of the stopper member.
36. The device of any of embodiments 24-35, wherein the stopper member includes a first portion and a second portion.
37. The device of any of embodiments 24-36, wherein the first portion of the stopper member contacts the second end of the inner tubular member.
38. The device of any of embodiments 24-37, wherein the second portion of the stopper member contacts a portion of the inner surface of the inner tubular member that is adjacent to the second opening of the inner tubular member such that the portion of the inner surface of the inner tubular member hugs the second portion of the stopper member.
39. The device of any of embodiments 24-38, wherein the opening of the stopper member and the second opening of the inner tubular member are concentric.
40. The device of any of embodiments 24-39, wherein the suction member further includes an extended central portion and a connecting portion, the connecting portion connecting the extended central portion to the handle portion, and a width of the extended central portion is larger than a diameter of an opening of the stopper member to prevent the suction member from being entirely pulled out of the inner tubular member.
41. The device of any of embodiments 24-40, wherein the end portion of the suction member includes a side surface and a top surface, and the side surface is parallel to a portion of the inner surface of the inner tubular member that is adjacent to the first opening of the inner tubular member.
42. The device of any of embodiments 24-41, wherein the side surface of the end portion of the suction member is cylindrical in shape, and the top surface of the end portion of the suction member is rounded.

What is claimed is:

1. A ligation device for treating hemorrhoids including a locking mechanism, comprising:
an inner tubular member having an inner surface, an outer surface, a first end, a second end, and a protrusion on the outer surface, the first end having a first opening and the second end having a second opening;
a suction member in the inner tubular member, the suction member including a handle portion and an end portion, and the handle portion allowing the end portion of the suction member to be pulled away from the first end of the inner tubular member;
a stopper member attached to the second end of the inner tubular member, the stopper member preventing the suction member from being entirely pulled out of the inner tubular member; and
an outer tubular member having an inner configuration corresponding to that of the inner tubular member, the outer tubular member including a notch that is configured to receive the protrusion of the inner tubular member, wherein a reception of the protrusion of the inner tubular member by a first portion of the notch of the outer tubular member allows movement of the outer tubular member toward the first end of the inner tubular member and a reception of the protrusion of the inner tubular member by a second portion of the notch of the outer tubular member prevents the movement of the outer tubular member toward the first end of the inner tubular member.

2. The ligation device of claim 1, wherein
the outer tubular member includes a handle, the handle forming a second axis extending away from a first axis formed by center points of the first opening and the second opening of the inner tubular member, and
the inner tubular member includes a tab, the tab forming a third axis extending away from the first axis formed by the center points of the first opening and the second opening of the inner tubular member.

3. The ligation device of claim 2, wherein the second axis and the third axis create an angle of about 0-45 degrees.

4. The ligation device of claim 1, wherein the reception of the protrusion of the inner tubular member by the second portion of the notch of the outer tubular member allows the outer tubular member to be rotated relative to the inner tubular member about a first axis formed by center points of the first opening and the second opening of the inner tubular member.

5. The ligation device of claim 1, wherein the reception of the protrusion of the inner tubular member by the second portion of the notch of the outer tubular member allows the inner tubular member to be rotated relative to the outer tubular member about a first axis formed by center points of the first opening and the second opening of the inner tubular member.

6. The ligation device of claim 1, wherein the protrusion on the outer surface of the inner tubular member is a button shaped protrusion.

7. The ligation device of claim 1, wherein a length of the protrusion extending away from the inner tubular member is greater than a thickness of the notch formed on the outer tubular member.

8. The ligation device of claim 1, wherein the first portion of the notch is parallel to a first axis formed by center points of the first opening and the second opening of the inner tubular member, and the second portion of the notch is perpendicular to the first portion.

9. The ligation device of claim 1, wherein the first portion of the notch and the second portion of the notch form an L-shape on the outer tubular member.

10. The ligation device of claim 1, wherein the end portion of the suction member includes a side surface and a top surface.

11. The ligation device of claim 10, wherein the side surface of the end portion of the suction member is substantially parallel to the inner surface of the inner tubular member that is adjacent to the first opening of the inner tubular member.

12. The ligation device of claim 10, wherein the side surface of the end portion of the suction member and the inner surface of the inner tubular member that is adjacent to the first opening of the inner tubular member form an angle of about 30 degrees or more.

13. A device, comprising:
an inner tubular member having an inner surface, an outer surface, a first end, a second end, and a protrusion on the outer surface, the first end having a first opening and the second end having a second opening;
a suction member in the inner tubular member, the suction member including a handle portion, and the handle portion allowing the suction member to be pulled toward and pushed away from the second end of the inner tubular member; and
an outer tubular member having an inner configuration corresponding to that of the inner tubular member, the outer tubular member including a notch that is configured to receive the protrusion of the inner tubular member, wherein a reception of the protrusion of the inner tubular member by a first portion of the notch of the outer tubular member allows movement of the outer tubular member toward the first end of the inner tubular member and a reception of the protrusion of the inner tubular member by a second portion of the notch of the outer tubular member prevents the movement of the outer tubular member toward the first end of the inner tubular member.

14. The device of claim 13, wherein
the outer tubular member includes a handle, the handle forming a second axis extending away from a first axis formed by center points of the first opening and the second opening of the inner tubular member, and
the inner tubular member includes a tab, the tab forming a third axis extending away from the first axis formed by the center points of the first opening and the second opening of the inner tubular member.

15. The device of claim 14, wherein the second axis and the third axis create an angle of about 0-45 degrees.

16. The device of claim 13, wherein
the reception of the protrusion of the inner tubular member by the second portion of the notch of the outer tubular member allows the outer tubular member to be rotated relative to the inner tubular member about a first axis formed by center points of the first opening and the second opening of the inner tubular member, and
the reception of the protrusion of the inner tubular member by the second portion of the notch of the outer tubular member allows the inner tubular member to be rotated relative to the outer tubular member about the first axis formed by the center points of the first opening and the second opening of the inner tubular member.

17. The device of claim 13, wherein the protrusion on the outer surface of the inner tubular member is a button shaped protrusion.

18. The device of claim 13, wherein a length of the protrusion extending away from the inner tubular member is greater than a thickness of the notch formed on the outer tubular member.

19. The device of claim 13, wherein the first portion of the notch is parallel to a first axis formed by center points of the first opening and the second opening of the inner tubular member, and the second portion of the notch is perpendicular to the first portion.

20. A device comprising:
an inner tubular member having an inner surface, an outer surface, a first end, a second end, and a protrusion on the outer surface, the first end having a first opening and the second end having a second opening;
a suction member in the inner tubular member, the suction member including a handle portion, and the handle portion allowing the suction member to be pulled toward and pushed away from the second end of the inner tubular member; and
an outer tubular member having an inner configuration corresponding to that of the inner tubular member, the outer tubular member including a notch that is configured to receive the protrusion of the inner tubular member, the notch including a wider portion and a narrower portion, the narrower portion of the notch preventing movement of the outer tubular member toward the first end of the inner tubular member.
